(19) United States
(12) Patent Application Publication
(10) Pub. No.: US 2014/0207168 A1
(43) Pub. Date: Jul. 24, 2014

(54) PUNCTURE APPARATUS

(71) Applicant: TERUMO KABUSHIKI KAISHA, Shibuya-ku (JP)

(72) Inventors: Masakatsu Kawaura, Kanagawa (JP); Shigeki Ariura, Kanagawa (JP)

(73) Assignee: TERUMO KABUSHIKI KAISHA, Shibuya-ku (JP)

(21) Appl. No.: 14/223,220

(22) Filed: Mar. 24, 2014

Related U.S. Application Data
(63) Continuation of application No. PCT/JP2012/070551, filed on Aug. 10, 2012.

(30) Foreign Application Priority Data
Sep. 27, 2011 (JP) 2011-211613

(51) Int. Cl. A61B 17/34

(52) U.S. Cl. CPC A61B 17/3403
USPC 606/185

(57) ABSTRACT

A puncture apparatus includes a puncture needle that punctures biological tissues; a longitudinal-shaped vaginal insertion member that is inserted into the vagina; a guiding member; an interlock member for interlocking the vaginal insertion member with the guiding member; an ultrasonic probe that is provided in a distal portion of the vaginal insertion member to transmit and receive ultrasonic waves; and an ultrasonic marker that is provided in the guiding member to be able to recognize an ultrasonic image which is obtained from the ultrasonic waves transmitted and received from the ultrasonic probe. The interlock member has a through-hole into which the puncture needle is inserted, and the through-hole is a supporting portion that movably supports the puncture needle to guide the distal end of the puncture needle to the ultrasonic marker.

### CROSS REFERENCES TO RELATED APPLICATIONS

This application claims priority as a continuation application under 35 U.S.C. §120 to International Application No. PCT/JP2012/070551 filed on Aug. 10, 2012, designating the U.S., and which claims priority to Japanese Application No. 2011-211613 filed on Sep. 27, 2011, the entire content of both of which is incorporated herein by reference.

### TECHNICAL FIELD

The disclosure herein relates to a puncture apparatus.

### BACKGROUND DISCUSSION

A patient suffering from urinary incontinence, and in particular, suffering from stress urinary incontinence, experiences urine leakage caused by intra-abdominal pressure occurring from normal exercise, laughing, coughing, sneezing or the like.

The fact that the pelvic floor muscles which support the urethra become weak after childbirth is cited as one of the causes.

There is a surgical treatment that is effective in treating urinary incontinence. During such surgical treatment, a tape-shaped implant referred to as a “sling” is indwelled inside the body to support the urethra (for example, as disclosed in JP-A-2010-099499).

In order to indwell the sling inside the body, an operator incises the vagina, dissects the area between the urethra and the vagina, and communicates the dissected region with the outside using a puncture needle or the like through an obturator foramen.

In such a state, the sling is indwelled inside the body.

However, there is a problem in that the procedure is very invasive and the patient bears a great burden in that the method in which the sling is indwelled uses a medical tool in the related art such as a surgical knife.

In addition, there is a concern that the urethra or the like will be damaged in the course of such a manual procedure by an operator or the operator himself or herself may also damage his or her fingertips.

### SUMMARY

The disclosure here provides a puncture apparatus with which a patient bears minimal burden and patient safety and operator safety are high.

An exemplary embodiment of the disclosure is directed to a puncture apparatus which includes a puncture needle that punctures biological tissues; a longitudinal-shaped vaginal insertion member that is inserted into the vagina; a guiding member; interlock means for interlocking the vaginal insertion member with the guiding member; an ultrasonic probe that is provided on one side of either a distal portion of the vaginal insertion member or the guiding member to transmit and receive ultrasonic waves; an ultrasonic marker that is provided on the other side of either the distal portion of the vaginal insertion member or the guiding member and can be recognized by an ultrasonic image which is obtained from the ultrasonic waves transmitted and received from the ultrasonic probe; and supporting means for movably supporting the puncture needle to guide the distal end of the puncture needle from a side of the ultrasonic probe toward the ultrasonic marker.

In the puncture apparatus according to an exemplary embodiment of the disclosure, the guiding member is preferably provided to be relatively movable in a longitudinal direction of the vaginal insertion member with respect to the vaginal insertion member and to be rotatable.

In addition, the supporting means preferably has heading direction changing means for changing direction in which the puncture needle advances in such a manner that the puncture needle faces a direction of the ultrasonic marker when the guiding member moves in the longitudinal direction of the vaginal insertion member relative to the vaginal insertion member or moves rotationally.

It is preferable, in the puncture apparatus according an exemplary embodiment of the disclosure, that the ultrasonic probe be provided in the distal portion of the vaginal insertion member and that the ultrasonic marker be provided in the guiding member.

Alternatively, it is also preferable that the ultrasonic probe be provided in the guiding member and that the ultrasonic marker be provided in the distal portion of the vaginal insertion member.

In the puncture apparatus according to an exemplary embodiment of the disclosure, it is preferable that the ultrasonic probe be rotatably supported by the guiding member.

The disclosure herein is further directed to a puncture apparatus including a puncture needle that punctures biological tissues; a longitudinal-shaped vaginal insertion member that is inserted into the vagina; a guiding member; interlock means for interlocking the vaginal insertion member with the guiding member; an ultrasonic probe that is provided on one side of either a distal portion of the vaginal insertion member or the guiding member to transmit and receive ultrasonic waves; an ultrasonic marker that is provided on the other side of either the distal portion of the vaginal insertion member or the guiding member to be able to recognize an ultrasonic image which is obtained from the ultrasonic waves transmitted and received from the ultrasonic probe; and supporting means for movably supporting the puncture needle to guide the distal end of the puncture needle from a side of the ultrasonic marker toward the ultrasonic probe.

Still further, according to an exemplary embodiment of the disclosure, the guiding member is preferably provided to be movable in the longitudinal direction of the vaginal insertion member relative to the vaginal insertion member.

Alternatively, the guiding member is preferably provided to be movable in the longitudinal direction of the vaginal insertion member relative to the vaginal insertion member and to be rotatable.

In the puncture apparatus according to the disclosure herein, the supporting means preferably has a through-hole or a groove into which the puncture needle is inserted.

In addition, according to the puncture apparatus disclosed here, the ultrasonic marker, which can be recognized by an ultrasonic image which is obtained from ultrasonic waves transmitted and received from the ultrasonic probe, is preferably provided in a distal portion of the puncture needle.

### BRIEF DESCRIPTION OF DRAWINGS

These and other features and advantages of the disclosure will become more readily apparent to those skilled in the art upon reading the following detailed description, in conjunction with the appended drawings in which:

FIG. 1(a)-1(d) discloses a first exemplary embodiment of a puncture apparatus according to the disclosure.

FIG. 2 describes the sequence of an operation of the puncture apparatus which is illustrated in FIG. 1.

FIG. 3 describes the sequence of the operation of the puncture apparatus which is illustrated in FIG. 1.

FIG. 4 describes the sequence of the operation of the puncture apparatus which is illustrated in FIG. 1.

FIG. 5 describes the sequence of the operation of the puncture apparatus which is illustrated in FIG. 1.

FIG. 6 describes the sequence of the operation of the puncture apparatus which is illustrated in FIG. 1.

FIG. 7 describes the sequence of the operation of the puncture apparatus which is illustrated in FIG. 1.

FIG. 8 describes the sequence of the operation of the puncture apparatus which is illustrated in FIG. 1.

FIG. 9 illustrates a second exemplary embodiment of the puncture apparatus according to the disclosure.

FIG. 10 is a side view illustrating a third exemplary embodiment of the puncture apparatus according to the disclosure.

FIG. 11 is a side view illustrating a fourth exemplary embodiment of the puncture apparatus according to the disclosure.

### DETAILED DESCRIPTION

A puncture apparatus according to the disclosure herein will be described in detail below with reference to exemplary embodiments which are illustrated in the accompanying drawings.

### First Embodiment

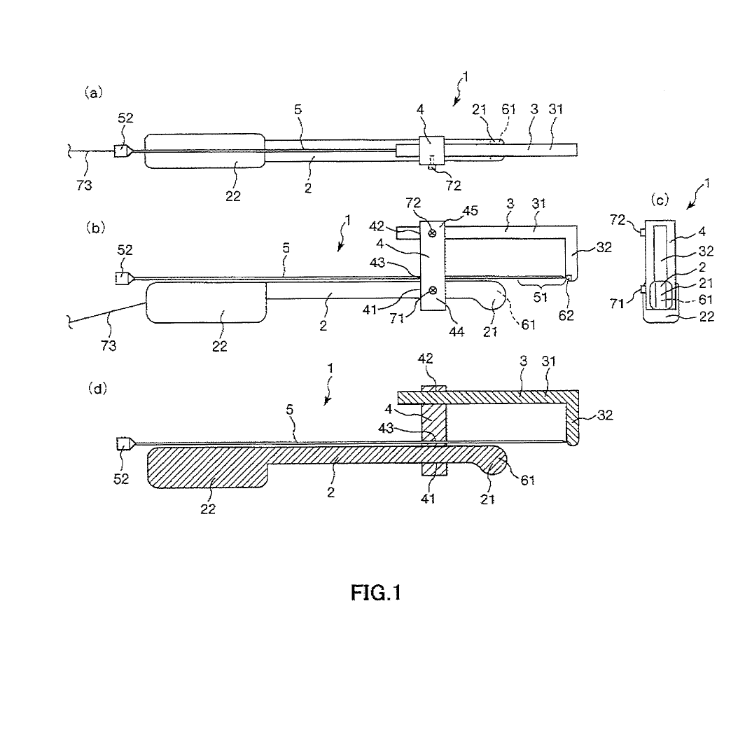

FIG. 1(a)-1(d) illustrate a first exemplary embodiment of the puncture apparatus according to the disclosure and, more particularly, FIG. 1 (a) is a plan view, FIG. 1 (b) is a side view, FIG. 1 (c) is a front view, and FIG. 1 (d) is a cross-sectional view thereof.

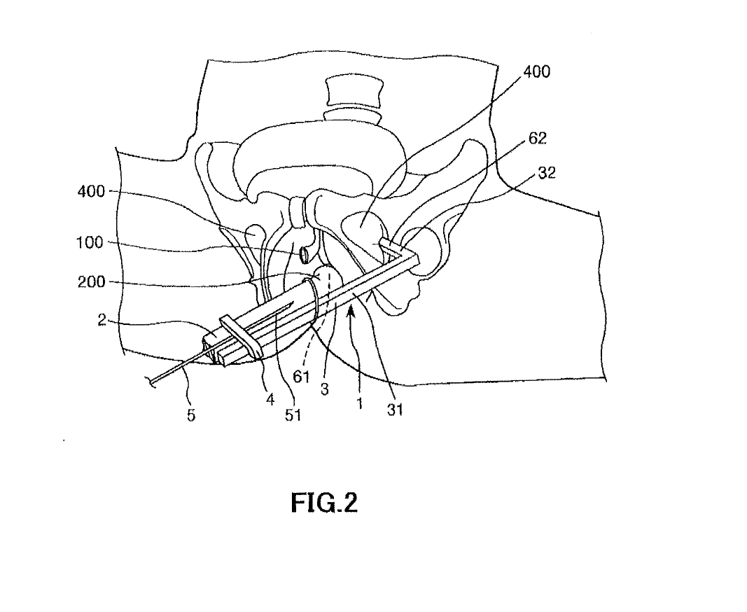

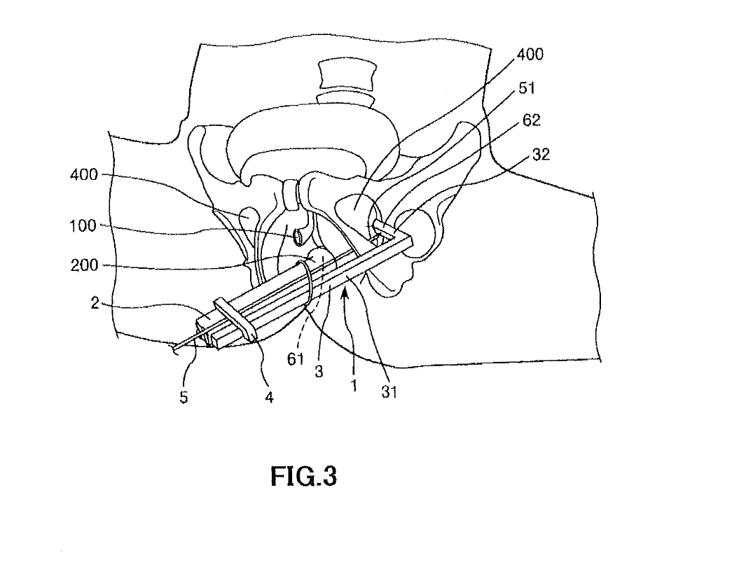

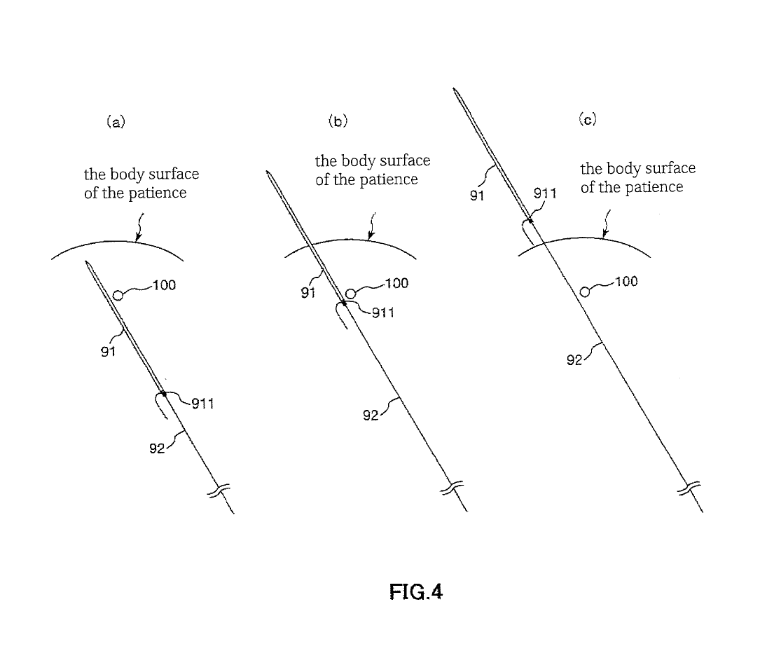

The description will be given based on the following premises: the right and left sides in FIGS. 1 (a), 1 (b) and 1 (d) indicate “distal ends” and “proximal ends”, respectively. In addition, the upper right and lower left sides in FIGS. 2 and 3 indicate “distal ends” and “proximal ends”, respectively. In addition, the upper right and upper left sides and lower left and lower right sides in FIGS. 4 to 8 indicate “distal ends” and “proximal ends”, respectively.

In FIG. 1 (d), the inside of an ultrasonic probe is not shown and is simply illustrated by a diagonal line for purposes of clarity.

A puncture apparatus 1 as illustrated in the drawings is an apparatus that is used for the treatment of urinary incontinence in women, and that is used to insert an implant (a device indwelled inside a living body) inside a living body.

The implant is an implantable device for the treatment of urinary incontinence in women, that is, a device that supports the urethra in a state where the urethra is pulled in a direction such that the urethra is separated from the vaginal wall.

For example, a long object that is flexible can be used as the implant.

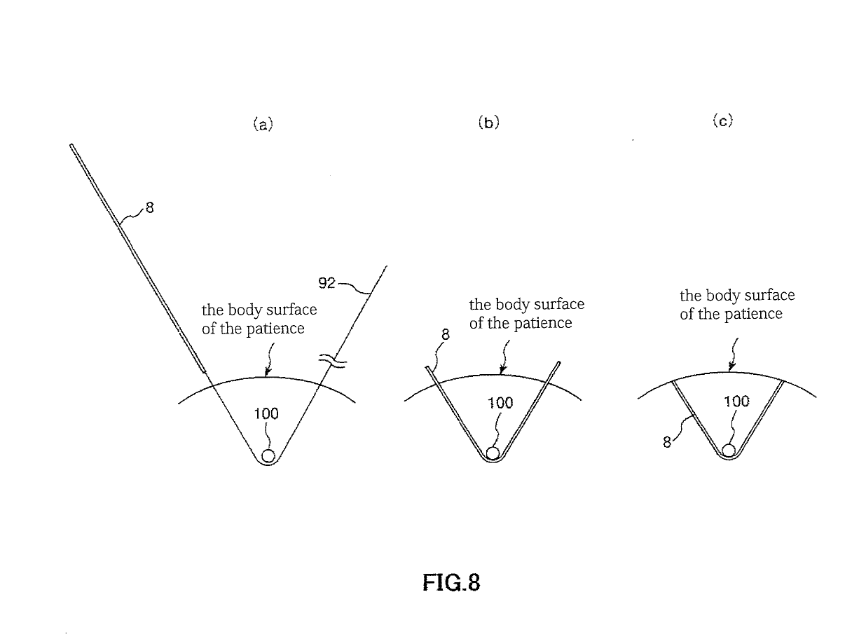

In the exemplary embodiment, the implant is configured to have a belt 8 (refer to FIG. 8 (c))

The belt 8 is referred to as a “sling”.

In addition, the dimension of the belt 8 is not particularly limited and is appropriately set, but the width thereof is preferably in a range of approximately 3 mm to 15 mm, and the thickness thereof is preferably in a range of approximately 0.2 mm to 2 mm.

The construction material of the belt 8 is not particularly limited, and, for example, various types of resin material with biocompatibility or the like can be used for the material of the belt 8.

In the exemplary embodiment as illustrated, the implant is configured to have one belt 8 but the disclosure here is not limited to the aforementioned configuration. For example, the implant may be configured to have a plurality of belts 8.

Further, the implant is not limited to the belt 8. For example, other long objects that are flexible such as a string or a cord can be used as the implant.

In a case where a string or a cord is used for the implant, when the cross-section thereof has a circular shape, the diameter thereof is preferably in a range of approximately 0.2 mm to 5 mm.

As illustrated in FIG. 1, the puncture apparatus 1 includes a puncture needle 5 that punctures biological tissues; a longitudinal-shaped vaginal insertion member 2 that is inserted into the vagina; a guiding member 3; an interlock member 4 defined by interlocking means for interlocking the vaginal insertion member 2 with the guiding member 3; an ultrasonic probe 61 that is provided in a distal portion of the vaginal insertion member 2 to transmit and receive ultrasonic waves; an ultrasonic marker 62 that is provided in the guiding member 3 to be able to recognize an ultrasonic image which is obtained from the ultrasonic waves transmitted and received from the ultrasonic probe 61.

Hereinafter, a member defined by the vaginal insertion member 2 and the ultrasonic probe 61 is simply referred to as the “vaginal insertion member 2”.

In addition, a member defined by of the guiding member 3 and the ultrasonic marker 62 is simply referred to as the “guiding member 3”.

In the exemplary embodiment of the disclosure described here, the guiding member 3 is arranged outside the body.

The puncture needle 5 has a linear shape.

In addition, the puncture needle 5 has a sharp needle tip at the distal end thereof, and a blade surface is formed in a distal portion thereof.

A grasping unit 52 is also formed in a proximal portion of the puncture needle 5.

The material of the puncture needle 5 is not particularly limited, and, for example, various types of metal materials such as stainless steel, aluminum, an aluminum alloy, titanium or a titanium alloy are cited as possible materials for the puncture needle 5.

An ultrasonic marker 51 is also provided in a distal portion of the puncture needle 5 to be able to recognize an ultrasonic image which is obtained from ultrasonic waves transmitted and received from the ultrasonic probe 61.

Accordingly, it is possible to conduct an operation while confirming the position where the distal end of the puncture needle 5 is directed by observing the ultrasonic image obtained by using the vaginal insertion member 2.

For example, a plurality of very small concaves (concave shapes) and convexes (convex shapes) or the like are provided as the ultrasonic marker 51.

That is, when the plurality of very small concaves and convexes are formed on the surface of the distal portion of the puncture needle 5, which is made of a metal material, a region where the concaves and convexes are formed can be detected by ultrasonic waves.

The puncture needle 5 may be a solid body or may have a tubular shape. The shape of the puncture needle 5 is also not limited to a linear shape. For example, the puncture needle 5 may be curved or bent.

The vaginal insertion member 2 has a head portion 21 at the distal portion thereof and a grasping unit 22 at a proximal portion thereof. The head portion 21 is a region that is inserted into the vagina of a patient to be attached to the vaginal wall, and the head portion 21 is rounded. Accordingly, patient safety can be improved.

In addition, the head portion 21 is provided with the ultrasonic probe 61 that has an ultrasonic vibrator or the like. For example, the ultrasonic vibrator has a configuration in which electrodes are formed on both surfaces of a piezoelectric substance made of PZT (lead zirconate titanate) or the like.

The ultrasonic vibrator of the ultrasonic probe 61 emits ultrasonic waves and receives reflective waves of the ultrasonic waves. That is, the ultrasonic vibrator transmits and receives ultrasonic waves. An image of an observation region can be captured by the transmission and reception of the ultrasonic waves.

More particularly, the ultrasonic vibrator transforms the received reflective waves of the ultrasonic waves into a signal, and the ultrasonic vibrator transmits via a cable 73 the signal to a control apparatus which is not illustrated. By way of example, the control apparatus receives the signal, processes the signal via a coordinate transformation process or the like, and generates an image signal for the observation region. The image signal is transmitted from the control apparatus to a display apparatus which is not illustrated, and the image of the observation region, that is, the ultrasonic image of the observation region is displayed on the display apparatus.

More simply put, the ultrasonic image is an image in which a target object is visualized by calculation of a distance to the target object and the like based on the time it takes from when the ultrasonic vibrator transmits the ultrasonic waves to when it receives the reflective waves thereof.

The shape of the interlock member 4 is not particularly limited by the disclosure, but the interlock member 4 has a rectangular shape in the exemplary embodiment.

Through-holes 41 and 42 and through-hole (supporting means) 43 are formed in the interlock member 4 along a left and right direction in FIG. 1 (b). Each of the through-holes 41 to 43 is formed in such a manner that directions of the central axes thereof coincide with each other, that is, the central axes thereof are parallel to each other.

In addition, the through-hole 41 is formed in a lower end portion of the interlock member 4 in FIG. 1 (b), the through-hole 42 is formed in an upper end portion of the interlock member 4 in FIG. 1 (b), the through-hole 43 is formed in the vicinity of the through-hole 41 between the through-holes 41 and 42 of the interlock member 4.

The vaginal insertion member 2 is inserted into the through-hole 41 of the interlock member 4 so as to be movable in a longitudinal direction of the vaginal insertion member 2.

The movement direction of the vaginal insertion member 2 is restricted by the through-hole 41, and the vaginal insertion member 2 is supported such that it is movable in the longitudinal direction relative to the interlock member 4.

In addition, the puncture apparatus 1 has a male screw 71, and a female screw portion 44 is configured to be screwed onto the male screw 71 in a region which corresponds to the through-hole 41 of the interlock member 4.

When the male screw 71 is rotated in a predetermined direction, the distal end of the male screw 71 is pressure-attached to the vaginal insertion member 2 to hinder the vaginal insertion member 2 from moving with respect to the interlock member 4.

When the male screw 71 is rotated in a direction opposite to the predetermined direction, the distal end of the male screw 71 is separated from the vaginal insertion member 2 to allow the vaginal insertion member 2 to move with respect to the interlock member 4.

A lock portion is thus configured to switch via the male screw 71 and the female screw portion 44 between a state where the vaginal insertion member 2 is movable with respect to the interlock member 4 and a state where the movement of the vaginal insertion member 2 is hindered.

In addition, the interlock member 4 is configured to have an adjustment mechanism that adjusts the separation distance between the head portion 21 of the vaginal insertion member 2, that is, the ultrasonic probe 61 and the ultrasonic marker 62 of the guiding member 3 which will be described later.

The shape of the guiding member 3 is not particularly limited according to the disclosure, but the guiding member 3 has an L shape in a side view, that is, FIG. 1(b) in the exemplary embodiment. In the L shape of the guiding member 3, a long region is referred to as a first region 31 and a short region is referred to as a second region 32.

The first region 31 of the guiding member 3 is inserted into the through-hole 42 of the interlock member 4 to be movable in a longitudinal direction of the first region 31.

Hence, a movement direction of the guiding member 3 is restricted by the through-hole 42, and the guiding member 3 is supported to be movable in the longitudinal direction relative to the interlock member 4. The direction in which the guiding member 3 moves coincides with a direction in which the vaginal insertion member 2 moves.

When a position of either the guiding member 3 or the vaginal insertion member 2 with respect to the interlock member 4 is adjusted or both positions are adjusted, a positional relationship between the vaginal insertion member 2 and the guiding member 3, that is, a separation distance between the head portion 21 of the vaginal insertion member 2 and the ultrasonic marker 62 of the guiding member 3 can be adjusted.

In addition, a lower end portion of the second region 32 of the guiding member 3 in FIG. 1 (b), that is, a distal portion of the second region 32 is a region that is attached to the body surface of the patient, and the distal portion of the second region 32 is rounded. Accordingly, patient safety can be improved.

The distal portion of the second region 32 is provided with the ultrasonic marker 62 that can be recognized by an ultrasonic image obtained from ultrasonic waves transmitted and received from the ultrasonic probe 61.

When the puncture needle 5, the movement direction of which is restricted by the through-hole 43 as described later, is moved in a direction of the distal end, a position of the ultrasonic marker 62 is set to be in a position facing the distal end of the puncture needle 5.

Accordingly, it is possible to conduct an operation, while confirming the position the distal end of the puncture needle 5 is directed, by observing the ultrasonic image obtained by using the ultrasonic probe 61.

For example, by using a metal material such as stainless steel, aluminum, an aluminum alloy, titanium or a titanium alloy as the construction material of the second region 32 of the guiding member 3 and providing a plurality of very small concaves and convexes on the surface thereof to define the ultrasonic marker 62.

That is, when the plurality of very small concaves and convexes are formed on the surface of a region made of a metal material, a region where the concaves and convexes are formed can be detected by ultrasonic waves.

The puncture apparatus 1 also has a male screw 72, and a female screw portion 45 configured to be screwed onto the male screw 72 formed in a region which corresponds to the through-hole 42 of the interlock member 4.

When the male screw 72 is rotated in a predetermined direction, the distal end of the male screw 72 is pressure-attached to the guiding member 3 to hinder the guiding member 3 from moving with respect to the interlock member 4.

In addition, when the male screw 72 is rotated in a direction opposite to the predetermined direction, the distal end of the male screw 72 is separated from the guiding member 3 to allow the guiding member 3 to move with respect to the interlock member 4.

A lock portion is thus configured to switch via the male screw 72 and the female screw portion 45 between a state where the guiding member 3 is movable with respect to the interlock member 4 and a state where movement of the guiding member 3 is hindered.

In addition, the puncture needle 5 is inserted into the through-hole 43 of the interlock member 4 for use and the puncture needle 5 inserted into the through-hole 43 is movable in a longitudinal direction thereof.

A movement direction of the puncture needle 5 is restricted by the through-hole 43, and the puncture needle 5 is supported to be movable in the longitudinal direction with respect to the interlock member 4.

A direction in which the through-hole 43 of the interlock member 4 moves, a direction in which the guiding member 3 moves, and a direction in which the vaginal insertion member 2 moves coincide with each other.

As mentioned before, when the puncture needle 5 moves in the direction of the distal end, the puncture needle 5 is guided by the through-hole 43, and the distal end of the puncture needle 5 faces the ultrasonic marker 62.

That is, the puncture needle 5 is movably supported in such a manner that the through-hole 43 guides the distal end of the puncture needle 5 to face the ultrasonic marker 62.

The puncture needle 5 may be inserted into the through-hole 43 in advance or the puncture needle 5 may be inserted into the through-hole 43 when used.

An exemplary sequence of an operation of the puncture apparatus 1, that is, a sequence taken when the implant defined by the belt 8 is implanted inside the living body will be described below.

One skilled in the art will recognize that an operator conducts a manual procedure while watching an ultrasonic image which is obtained from ultrasonic waves transmitted and received from the ultrasonic probe 61 of the puncture apparatus 1 and which is displayed on the display apparatus.

First, two puncture holes are formed in a patient by the puncture apparatus 1.

One of the two puncture holes is through the body surface from the inside of a vagina 200 via an obturator foramen 400 on the right side in FIG. 2, and the other of the two puncture holes is through the body surface from the inside of the vagina 200 via an obturator foramen 400 on the left side in FIG. 2.

Note that, since the manual procedures by which the two puncture holes are formed are the same, hereinafter, the formation of the puncture hole that is through the body surface from the inside of the vagina 200 via the right-side obturator foramen 400 in FIG. 2 will be representatively described.

First, in a state where the puncture needle 5 is not inserted into the through-hole 43 of the interlock member 4 of the puncture apparatus 1, the vaginal insertion member 2 is inserted into the vagina 200 of the patient, and the guiding member 3 is arranged outside the patient body.

The distal portion of the second region 32 of the guiding member 3 is pushed to the right-side inguinal region of the patient in FIG. 2, or a region in the vicinity thereof that is, a position which corresponds to the right-side obturator foramen 400 in FIG. 2.

At this time, as necessary, a position of either the vaginal insertion member 2 or the guiding member 3 or positions of both are changed with respect to the interlock member 4, to adjust the positional relationship between the vaginal insertion member 2 and the guiding member 3.

That is, as necessary, the male screw 71 is rotated in a loosening direction to move the vaginal insertion member 2 in the direction of the distal end or the direction of the proximal end with respect to the interlock member 4. The male screw 71 is rotated in a tightening direction to hinder the vaginal insertion member 2 from moving with respect to the interlock member 4.

In addition, as necessary, the male screw 72 is rotated in a loosening direction to move the guiding member 3 in the direction of the distal end or the direction of the proximal end with respect to the interlock member 4. The male screw 72 is rotated in a tightening direction to hinder the guiding member 3 from moving with respect to the interlock member 4.

Subsequently, as illustrated in FIG. 2, the puncture needle 5 is inserted into the through-hole 43 of the guiding member 3, and as illustrated in FIG. 3, the puncture needle 5 is moved in the direction of the distal end, is inserted into the vagina 200 and is further moved in the direction of the distal end.

Since the through-hole 43 guides the puncture needle 5 in such a manner that the distal end of the puncture needle 5 faces the ultrasonic marker 62 of the guiding member 3, a urethra 100 is protected from being punctured by the puncture needle 5 and thus, is safe.

More particularly, the puncture needle 5 is guided by the through-hole 43 to puncture the vaginal wall from the inside of the vagina 200, to avoid the urethra 100, to be inserted through the obturator foramen 400, to puncture the biological tissues from the obturator foramen 400 to the body surface, and to protrude outside from the inguinal region or a region in the vicinity of the inguinal region.

Further, since the operator can conduct a manual procedure while visually recognizing the ultrasonic marker 51 that points to the distal portion of the puncture needle 5; the ultrasonic marker 62 of the guiding member 3 which points to a position which the distal end of the puncture needle 5 faces; the urethra; blood vessels; the obturator foramen; and the like via the ultrasonic image, it is possible to minutely adjust the puncture needle 5 in such a manner that the urethra 100 or the blood vessels are not punctured and to more safely make a puncture with the puncture needle 5.

By using the puncture apparatus 1, it is also possible for the operator himself or herself to prevent puncturing his or her finger tips by the puncture needle 5 and thus, it is safer for the operator.

Subsequently, the puncture needle 5 is removed.

As necessary, similar to the above description, a position of either the vaginal insertion member 2 or the guiding member 3 or positions of both are changed with respect to the interlock member 4, to adjust the positional relationship between the vaginal insertion member 2 and the guiding member 3 and to detach the puncture apparatus 1 from the patient.

In this way, the patient has the puncture hole that is through the body surface from the inside of the vagina 200 via the right-side obturator foramen 400 in FIG. 3.

Similar to the above description, the second puncture hole, which is through the body surface from the inside of the vagina 200 via the left-side obturator foramen 400 in FIG. 3, is formed.

Note that any one of the two puncture holes may be formed in advance of the other.

Subsequently, as illustrated in FIG. 4 (a), a guide wire 92 goes through a hole 911 of a needle 91 that has the hole 911 in a proximal portion thereof, and a distal portion of the guide wire 92 is separably connected to the proximal portion of the needle 91.

The shape of the needle 91 is not particularly limited, and, for example, a linear shape, a curved shape, or the like is cited as exemplary shapes of the needle 91. In addition, the needle 91 may be a solid body or may have a tubular shape.

Subsequently, the needle 91 is inserted into either the puncture hole via the left-side obturator foramen 400 in FIG. 3 or the puncture hole via the right-side obturator foramen 400 in FIG. 3.

Hereinafter, a case where the needle 91 is inserted first into the puncture hole via the left-side obturator foramen 400 in FIG. 3 will be described, but it will be apparent to one skilled in the art that the needle 91 may be inserted first into the puncture hole via the right-side obturator foramen 400 in FIG. 3.

As illustrated in FIG. 4 (a), the needle 91 is inserted into the puncture hole via the left-side obturator foramen 400 in FIG. 3, and as illustrated in FIG. 4 (b), the needle 91 is moved in the direction of the distal end, and as illustrated in FIG. 4 (c), the needle 91 is pulled out of the puncture hole.

The distal portion of the guide wire 92 is then separated from the proximal portion of the needle 91.

Subsequently, biological tissues between the urethra 100 and the vaginal wall and the like are dissected using a dissection tool which is not illustrated, to form a space into which the belt 8 is inserted.

Note that the dissection tool is moved to a target position along the guide wire 92.

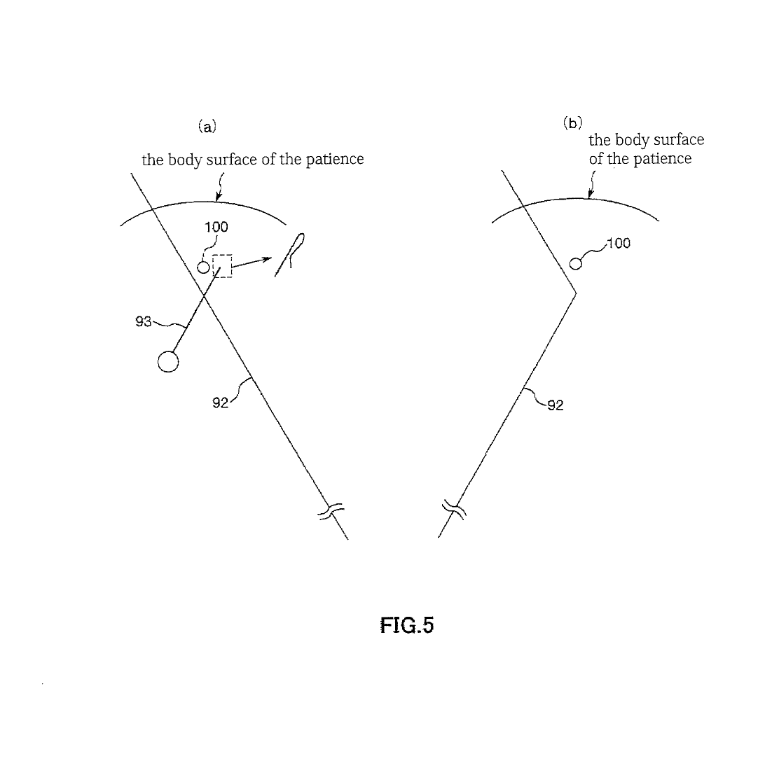

As illustrated in FIG. 5 (a), a wire hook tool 93 is inserted into the puncture hole via the right-side obturator foramen 400 in FIG. 3, the guide wire 92 is hooked by the wire hook tool 93, and the wire hook tool 93 is moved in the direction of the proximal end.

Subsequently, as illustrated in FIG. 5 (b), a proximal side of the guide wire 92 is pulled out through the puncture hole to move outside the vagina.

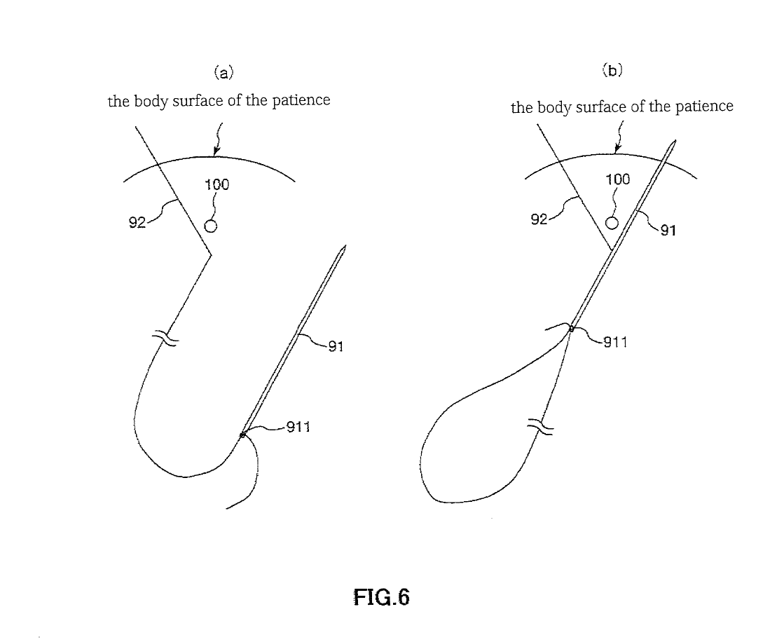

As illustrated in FIG. 6 (a), the guide wire 92 goes through the hole 911 of the needle 91, and a proximal portion of the guide wire 92 is separably connected to the proximal portion of the needle 91.

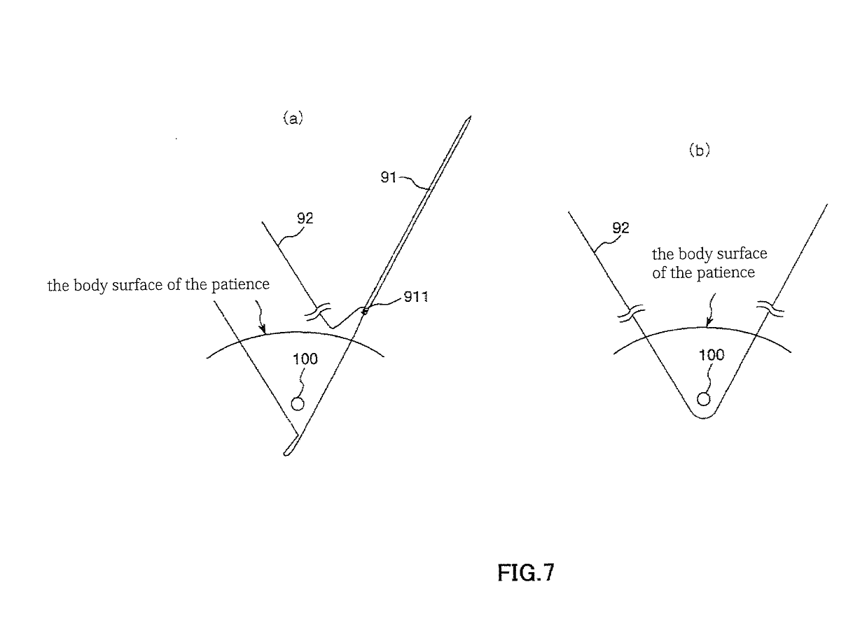

Subsequently, as illustrated in FIG. 6 (b), the needle 91 is inserted into the puncture hole via the right-side obturator foramen 400 in FIG. 3, and the needle 91 is moved in the direction of the distal end, and as illustrated in FIG. 7 (a), the needle 91 is pulled out of the puncture hole.

As illustrated in FIG. 7 (a), the proximal portion of the guide wire 92 is separated from the proximal portion of the needle 91.

Accordingly, in a state where the urethra 100 is hooked by the guide wire 92, one end portion of the guide wire 92 protrudes outside the body via one puncture hole, and the other end portion thereof protrudes outside the body via the other puncture hole.

As illustrated in FIG. 8 (a), one end portion of the belt 8 is separably connected to one end portion of the guide wire 92, and the other end portion of the guide wire 92 is pulled.

Accordingly, as illustrated in FIG. 8 (b), the guide wire 92 and the belt 8 move, and in a state where the urethra 100 is hooked by the belt 8, one end portion of the belt 8 protrudes outside the body via one puncture hole, and the other end portion thereof protrudes outside the body via the other puncture hole. Subsequently, each of both end portions of the belt 8 is pulled by a predetermined force.

Accordingly, the urethra 100 is pulled by the tension from the belt 8 in a direction in which the urethra 100 is separated from the vaginal wall, and the urethra 100 is supported by the belt 8.

As illustrated in FIG. 8 (c), unnecessary portions of the belt 8 are then excised, and a predetermined suture and the like are done to finish a manual procedure.

As described above, according to an exemplary embodiment of the puncture apparatus 1, when the implant is indwelled, since only low invasive manual procedures such as punctures by the puncture needle 5 can accomplish the indwelling of the implant, and an incision with high invasion and the like may not be required, patient burden is minimal and patient safety is improved.

In addition, when the operator punctures the living body using the puncture needle 5, the operator can conduct a manual procedure while visually recognizing the ultrasonic marker 62, the urethra, blood vessels, the obturator foramen and the like by observing the ultrasonic image, and the distal end of the puncture needle 5 is guided to the ultrasonic marker 62 so the urethra 100 can be protected from being punctured by the puncture needle 5 and thus, is safe.

In addition, it is possible for the operator himself or herself to prevent puncturing his or her finger tips by the puncture needle 5 and thus, it is safer for the operator.

In the first exemplary embodiment discussed above, the through-hole 43 is used as the supporting means that guides the puncture needle 5, but the disclosure here is not limited to such embodiment. For example, a groove and the like may be used as a supporting portion.

In addition, in the first exemplary embodiment, each of the vaginal insertion member 2 and the guiding member 3 is configured to be movable with respect to the interlock member, but the disclosure herein is not limited to such an embodiment. Either of the vaginal insertion member 2 and the guiding member 3 may be configured to be movable with respect to the interlock member. Or alternatively, neither the vaginal insertion member 2 nor the guiding member 3 may be configured to be movable.

In addition, in the first exemplary embodiment, the puncture holes formed in the patient by the puncture needle 5 are through-holes, but the disclosure is not limited to the same. The puncture holes may not be through-holes.

### Second Embodiment

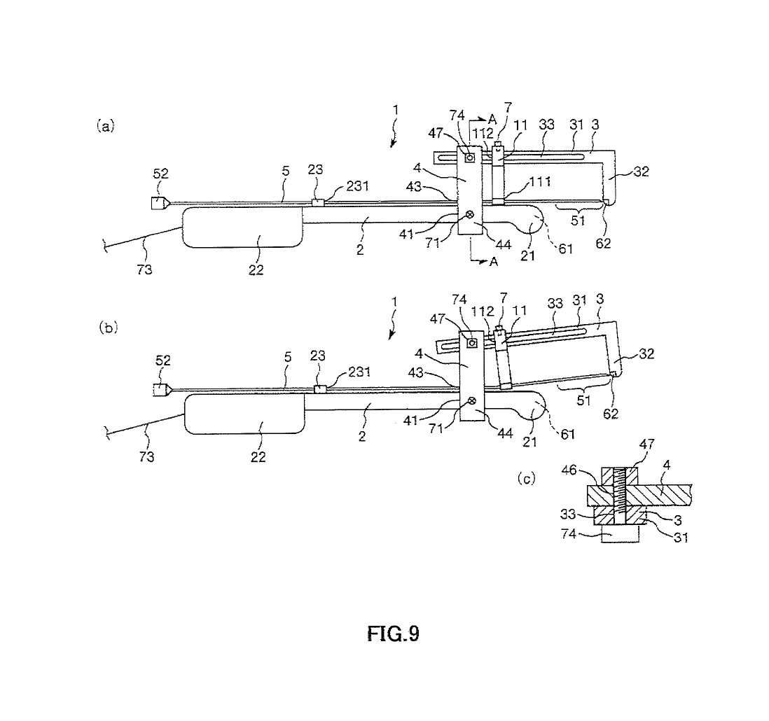

FIGS. 9(a)-9(c) illustrate a second exemplary embodiment of the puncture apparatus according to the disclosure and, more particularly, FIGS. 9 (a) and 9 (b) are side views, and FIG. 9 (c) is a cross-sectional view taken along A-A line in FIG. 9 (a).

Hereinafter, description will be given on the premise that the left and right sides in FIGS. 9 (a) and 9 (b) indicate “distal ends” and “proximal ends”, respectively.

The second exemplary embodiment will be described with the focus on the differences from the aforementioned first exemplary embodiment, and the description of the same matters will be omitted.

As illustrated in FIGS. 9(a)-9(c), in the puncture apparatus 1 according to the second exemplary embodiment, the guiding member 3 is provided to be relatively movable in the longitudinal direction of the vaginal insertion member 2 with respect to the vaginal insertion member 2 and to be rotatable.

In the configuration which is illustrated, the guiding member 3 is supported to be movable in the longitudinal direction of the vaginal insertion member 2 and to be rotatable by the interlock member 4.

Regarding the specific configuration, a long hole 33 is formed in the first region 31 of the guiding member 3. The long hole 33 is extended along a longitudinal direction of the first region 31.

In addition, a through-hole 46 is formed in an upper end portion of the interlock member 4, the through-hole 46 being formed along a vertical direction as shown in FIG. 9 (c).

In addition, the puncture apparatus 1 has a male screw 74, and a female screw portion 47, configured to be screwed onto the male screw 74 and communicating with the through-hole 46, is formed in a region which corresponds to the upper-side through-hole 46 of the interlock member 4.

In a state where the long hole 33 of the guiding member 3 and the through-hole 46 of the interlock member 4 communicate with each other, the male screw 74 is inserted into the long hole 33 and the through-hole 46 from below, as shown in FIG. 9(c), to thereby screw into the female screw portion 47.

Accordingly, the guiding member 3 is rotatable around the male screw 74 inserted into the long hole 33.

In addition, the long hole 33 and the male screw 74 restrict a movement direction of the guiding member 3 in a direction in which the long hole 33 is formed, and the guiding member 3 is movable with respect to the interlock member 4.

When the male screw 74 is rotated in a predetermined direction, the male screw 74 is tightened, and the guiding member 3 is interposed between the male screw 74 and the interlock member 4, and thus the guiding member 3 can be hindered from moving and rotationally moving with respect to the interlock member 4.

In addition, when the male screw 74 is rotated in a direction opposite to the predetermined direction, the male screw 74 is loosened, and thus the guiding member 3 is movable and rotatable with respect to the interlock member 4.

A lock portion is thus configured to switch via the male screw 74 and the female screw portion 47 between a state where the guiding member 3 is movable and rotatable with respect to the interlock member 4 and a state where movement and rotary movement of the guiding member 3 are hindered.

In addition, the supporting means of the puncture apparatus 1 has a heading direction changing member 11 as heading direction changing means for changing direction in which the puncture needle 5 advances in such a manner that the puncture needle 5 faces a direction of the ultrasonic marker 62 when the guiding member 3 moves in the longitudinal direction of the vaginal insertion member 2 relative to the vaginal insertion member 2 or rotationally moves.

The heading direction changing member 11 is a member that interlocks the guiding member 3 with the puncture needle 5, and each of the guiding member 3 and the puncture needle 5 is movably supported by the heading direction changing member 11.

The shape of the heading direction changing member 11 is not particularly limited, but in the exemplary embodiment, the heading direction changing member 11 has a rectangular shape. The thickness of each of the upper and lower end portions of the heading direction changing member 11 shown in FIG. 9 (a), that is, the length of each end portion in a direction perpendicular to the FIG. 9 (a) drawing sheet is set to be thicker than that of a center portion thereof.

Through-holes 111 and 112 are formed in the lower and upper end portions, respectively, of the heading direction changing member 11. Each of the through-holes 111 and 112 is formed along a left and right direction in FIG. 9 (a).

In addition, as illustrated in FIG. 9 (a), in a state where directions of the central axes of each of the through-holes 111 and 112 coincide with each other, that is, the central axes thereof are parallel to each other and the axis line of the heading direction changing member 11 is parallel to the axis line of the interlock member 4, each of the through-holes 111 and 112 is formed in such a manner that the central axis of the through-hole 111 is positioned in an extension line of the central axis of a through-hole 231 of a supporting portion 23 to be described later and the central axis of the through-hole 112 intersects the central axis of the male screw 74.

The first region 31 of the guiding member 3 is inserted into the through-hole 112 of the heading direction changing member 11 to be movable in the longitudinal direction of the first region 31. In other words, the heading direction changing member 11 is supported by the first region 31 to be movable along the longitudinal direction of the first region 31.

In addition, as shown in FIG. 9 (a), the vaginal insertion member 2 has the supporting portion 23 on an upper side, and the supporting portion 23 supports the puncture needle 5 in such a manner that the puncture needle 5 is movable along the longitudinal direction thereof.

The through-hole 231 is formed in the supporting portion 23 extending along the left and right direction relative to the drawing in FIG. 9 (a).

The puncture needle 5 is inserted into the through-hole 231 of the vaginal insertion member 2, the through-hole 43 of the interlock member 4 and the through-hole 111 of the heading direction changing member 11 for use. The puncture needle 5, when inserted into each of the through-holes 231, 43 and 111, is movable in the longitudinal direction thereof.

In the puncture apparatus 1, as illustrated in FIG. 9 (a), in a state where the axis line of the heading direction changing member 11 is parallel to the axis line of the interlock member 4, the axis line of the first region 31 of the guiding member 3 is parallel to the axis line of the puncture needle 5.

When the puncture needle 5 is moved in the direction of the distal end, the puncture needle 5 is guided by the through-hole 231 of the supporting portion 23 and the through-hole 111 of the heading direction changing member 11, and the distal end of the puncture needle 5 faces the ultrasonic marker 62.

In addition, as illustrated in FIG. 9 (b), when the guiding member 3 is rotated counterclockwise by a predetermined amount, the heading direction changing member 11 slopes at an angle which corresponds to the rotation amount, and the distal portion of the puncture needle 5 is bent, in the upper right direction as shown in FIG. 9 (b), by the heading direction changing member 11.

The bent angle of the distal portion of the puncture needle 5 is an angle which corresponds to the rotation amount of the guiding member 3, and the heading direction changing member 11 retains a state where the axis line of the first region 31 of the guiding member 3 is parallel to the axis line of a region on the distal side of the puncture needle 5 beyond the through-hole 111.

Accordingly, when the puncture needle 5 is moved in the direction of the distal end, the puncture needle 5 is guided by the through-hole 111, and the distal end of the puncture needle 5 faces the ultrasonic marker 62.

When the guiding member 3 is rotationally moved, a heading direction of the puncture needle 5 is changed in such a manner that the puncture needle 5 faces the direction of the ultrasonic marker 62.

Preferably, the main part of the supporting means is configured to include the through-holes 231 and 111.

In addition, as illustrated in FIG. 9 (b), when the guiding member 3 is moved in the longitudinal direction with respect to the interlock member 4 in a state where the distal portion of the puncture needle 5 is bent, the heading direction changing member 11 is moved in the longitudinal direction with respect to the guiding member 3 in such a manner that the heading direction changing member 11 is not moved with respect to the interlock member 4.

Accordingly, the heading direction changing member 11 can retain a state where the axis line of the first region 31 of the guiding member 3 is parallel to the axis line of the region on the distal side of the puncture needle 5 beyond the through-hole 111.

Thus, when the puncture needle 5 is moved in the direction of the distal end, the puncture needle 5 is guided by the through-hole 111, and the distal end of the puncture needle 5 faces the ultrasonic marker 62.

When the guiding member 3 is moved in the longitudinal direction with respect to the interlock member 4 in the state as illustrated in FIG. 9 (a), the heading direction changing member 11 is not necessarily required to be moved in the longitudinal direction with respect to the guiding member 3 and hence, the heading direction changing member 11 is not necessarily moved with respect to the interlock member 4.

According to the puncture apparatus 1 of the second exemplary embodiment, the same effects as in the aforementioned first exemplary embodiment are obtained.

In the puncture apparatus 1, since the guiding member 3 is configured to be movable in the longitudinal direction and rotatable with respect to the interlock member 4 and the direction in which the puncture needle 5 advances can be changed, it is possible to handle the individual differences among patients or various types of disease cases and to more reliably prevent the urethra from being punctured.

### Third Embodiment

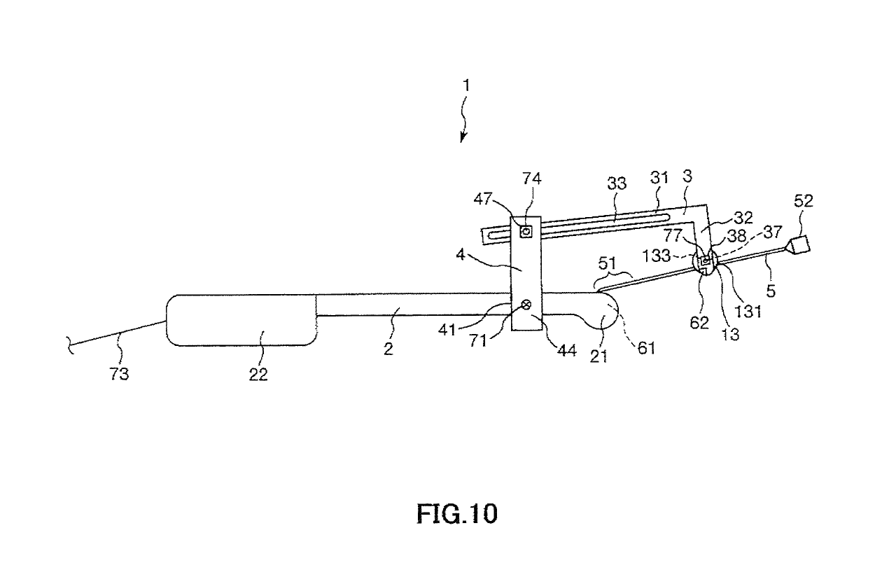

FIG. 10 is a side view illustrating a third exemplary embodiment of the puncture apparatus according to the disclosure.

Hereinafter, description will be given on the premise that the left and right sides in FIG. 10 indicate a “distal end” and a “proximal end”, respectively.

The third exemplary embodiment will be described with the focus on the differences from the aforementioned second exemplary embodiment, and the description of the same matters will be omitted.

As illustrated in FIG. 10, in the puncture apparatus 1 according to the third exemplary embodiment, the puncture needle 5 is configured to be rotatable with respect to the guiding member 3 and to be moved in an axial direction of the puncture needle 5.

That is, the puncture apparatus 1 has a supporting member 13 which supports the puncture needle 5 in such a manner that the puncture needle 5 is movable in the axial direction thereof, and the supporting member 13 is rotatably provided in the lower end portion of the second region 32 of the guiding member 3 as shown in FIG. 10, that is, in the distal portion of the second region 32.

The shape of the supporting member 13 is not particularly limited, but in the exemplary embodiment, the supporting member 13 has a columnar shape or a disc-like shape.

The supporting member 13 is arranged in such a manner that the central axis thereof faces a direction perpendicular to the FIG. 10 drawing sheet.

Each of a through-hole (supporting means) 131 and a through-hole 133 is formed in the supporting member 13.

The through-hole 133 is formed in a direction perpendicular to the FIG. 10 drawing sheet, that is, is formed along the central axis of the supporting member 13.

In addition, the through-hole 131 is formed at a position offset from the through-hole 133 along a direction parallel to the FIG. 10 drawing sheet.

The puncture needle 5 is inserted into the through-hole 131 of the supporting member 13 for use, and the puncture needle 5 as inserted into the through-hole 131 is movable in the longitudinal direction thereof. That is, the through-hole 131 movably supports the puncture needle 5 in such a manner that the puncture needle 5 protrudes from the position of the ultrasonic marker 62 or a position in the vicinity of the ultrasonic marker 62 and the distal end of the puncture needle 5 is guided to the ultrasonic probe 61.

In addition, a through-hole 37 is formed in the lower end portion of the second region 32 of the guiding member 3 as shown in FIG. 10. The through-hole 37 is formed along a direction perpendicular to the FIG. 10 drawing sheet.

In addition, the puncture apparatus 1 has a male screw 77, and a female screw portion 38, configured to be screwed onto the male screw 77 and communicating with the through-hole 37, is formed at a position which corresponds to the through-hole 37 on a front side of the FIG. 10 drawing sheet in the second region 32 of the guiding member 3.

In a state where the through-hole 37 of the guiding member 3 and the through-hole 133 of the supporting member 13 are communicated with each other, the male screw 77 is inserted into the through-holes 133 and 37 from a back side of the FIG. 10 drawing sheet to be screwed into the female screw portion 38.

Accordingly, the supporting member 13 is rotatable around the male screw 77.

When the male screw 77 is rotated in a predetermined direction, the male screw 77 is tightened, and the supporting member 13 is interposed between the male screw 77 and the guiding member 3, and thus the supporting member 13 can be hindered from rotationally moving with respect to the guiding member 3.

In addition, when the male screw 77 is rotated in a direction opposite to the predetermined direction, the male screw 77 is loosened, and thus the supporting member 13 is rotatable with respect to the guiding member 3.

A lock portion is thus configured to switch via the male screw 77 and the female screw portion 38 between a state where the supporting member 13 is rotatable with respect to the guiding member 3 and a state where rotary movement of the supporting member 13 is hindered.

According to the puncture apparatus 1, the same effects as in the aforementioned second exemplary embodiment are obtained.

In the third exemplary embodiment, the supporting member 13 is rotatably supported by the guiding member 3, but the disclosure is not limited to such an embodiment. The supporting member 13 may be fixedly provided in the guiding member 3.

In addition, the guiding member 3 may serve as the supporting member 13. That is, the guiding member 3 may be provided with a through-hole in which the puncture needle 5 is inserted and which supports the puncture needle 5 in such a manner that the puncture needle 5 is movable in the longitudinal direction thereof.

### Fourth Embodiment

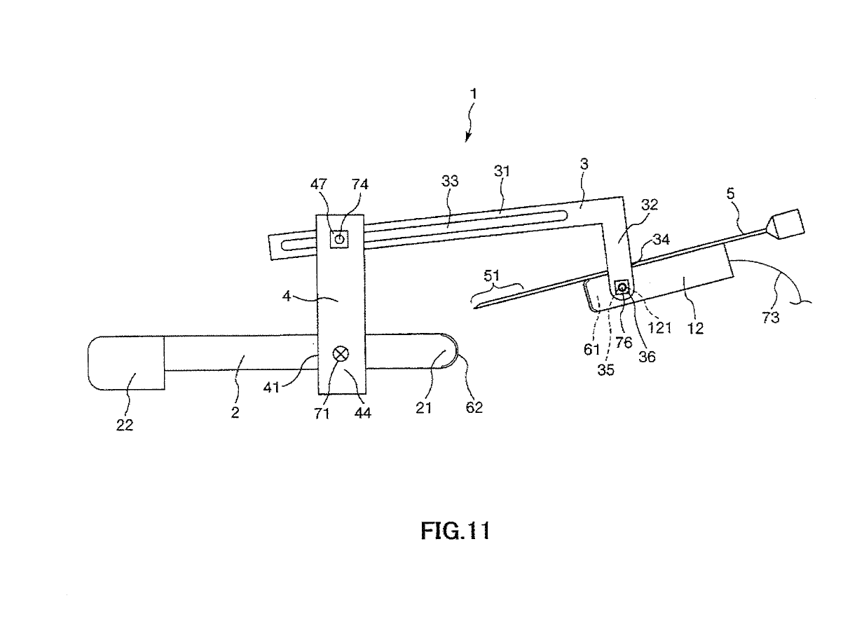

FIG. 11 is a side view illustrating a fourth exemplary embodiment of the puncture apparatus according to the disclosure.

Hereinafter, description will be given on the premise that the left and right sides in FIG. 11 indicate a “distal end” and a “proximal end”, respectively.

The fourth exemplary embodiment will be described with the focus on the differences from the aforementioned third exemplary embodiment, and the description of the same matters will be omitted.

As illustrated in FIG. 11, in the puncture apparatus 1 according to the fourth exemplary embodiment, the ultrasonic probe 61 is provided in the guiding member 3. That is, the ultrasonic probe 61 is rotatably supported by the guiding member 3.

In addition, the ultrasonic marker 62 is provided in the distal portion of the vaginal insertion member 2, that is, in the head portion 21.

The puncture needle 5 is supported to be movable in the longitudinal direction of the vaginal insertion member 2 by the guiding member 3.

In a state as illustrated in FIG. 11, the axis line of the puncture needle 5 slopes at a predetermined angle with respect to the axis line of the vaginal insertion member 2.

The puncture apparatus 1 has an attachment member 12. The shape of the attachment member 12 is not particularly limited, but in the exemplary embodiment, the attachment member 12 has a rectangular shape.

A left end portion of the attachment member 12 is a region that is attached to the body surface of a patient in FIG. 11, and the attachment member 12 is rounded. Accordingly, patient safety can be improved.

The ultrasonic probe 61 is provided in the left end portion of the attachment member 12 in FIG. 11. Hereinafter, the left end portion of the attachment member 12 in FIG. 11 is referred to as a “distal portion”.

In addition, a through-hole 121 is formed in the distal portion of the attachment member 12. The through-hole 121 is formed along a direction perpendicular to the FIG. 11 drawing sheet.

Similarly, a through-hole 35 is formed in the lower end portion of the second region 32 of the guiding member 3 in FIG. 11. The through-hole 35 is formed along the direction perpendicular to the FIG. 11 drawing sheet.

In addition, the puncture apparatus 1 has a male screw 76, and a female screw portion 36, configured to be screwed onto the male screw 76 and communicating with a through-hole 35, is formed at a position which corresponds to the through-hole 35 on a front side of the FIG. 11 drawing sheet in the second region 32 of the guiding member 3.

In a state where the through-hole 35 of the guiding member 3 and the through-hole 121 of the attachment member 12 are communicated with each other, the male screw 76 is inserted into the through-holes 121 and 35 from a back side of the FIG. 11 drawing sheet to be screwed into the female screw portion 36.

Accordingly, the attachment member 12 is rotatable around the male screw 76.

When the male screw 76 is rotated in a predetermined direction, the male screw 76 is tightened, and the attachment member 12 is interposed between the male screw 76 and the guiding member 3, and thus the attachment member 12 can be hindered from rotationally moving with respect to the guiding member 3.

In addition, when the male screw 76 is rotated in a direction opposite to the predetermined direction, the male screw 76 is loosened, and thus the attachment member 12 is rotatable with respect to the guiding member 3.

A lock portion is thus configured to switch via the male screw 76 and the female screw portion 36 between a state where the attachment member 12 is rotatable with respect to the guiding member 3 and a state where rotary movement of the attachment member 12 is hindered.

In addition, a through-hole (supporting means) 34 is formed in the lower end portion of the second region 32 of the guiding member 3 in FIG. 11 along a left and right direction as shown in FIG. 11. The central axis of the through-hole 34 slopes at a predetermined angle with the axis line of the first region 31 of the guiding member 3.

The puncture needle 5 is inserted into the through-hole 34 of the guiding member 3 for use, and the puncture needle 5 when inserted into the through-hole 34 is movable in the longitudinal direction thereof.

That is, the through-hole 34 movably supports the puncture needle 5 in such a manner that the distal end of the puncture needle 5 is guided to the ultrasonic marker 62.

According to the puncture apparatus 1, the same effects as in the aforementioned third exemplary embodiment are obtained.

In the fourth exemplary embodiment, the ultrasonic probe 61 is rotatably supported by the guiding member 3, but the disclosure is not limited to such an embodiment. The ultrasonic probe 61 may be fixedly provided in the guiding member 3.

The puncture apparatus according to the disclosure herein has been described as above with reference to the exemplary embodiments which are illustrated, but the disclosure is not limited to the exemplary embodiments. The configuration of each portion can be replaced with any configuration having the same function.

In addition, any other configuration components may be added to the puncture apparatus according to the disclosure here.

The disclosure may be configured to arbitrarily combine two or more configurations of the exemplary embodiments.

The puncture apparatus of the disclosure may be configured in such a manner that the vaginal insertion member 2 is detached from the interlock member 4.

In addition, the puncture apparatus of the disclosure here may have protrusion-length adjustment means for adjusting the maximum protrusion length by which the puncture needle 5 protrudes from the interlock member 4 in the direction of the distal end.

Accordingly, for example, in a case where the puncture needle 5 makes a puncture hole that is not through a patient body, the maximum protrusion length of the puncture needle 5 is adjusted by the protrusion-length adjustment means, and thus it is possible to easily and reliably form the puncture hole of a desired length.

In the exemplary embodiments, cases are described where the puncture apparatus according to the disclosure here is applied to an apparatus used to implant an implantable implant inside the living body for the treatment of urinary incontinence in women, but the use of the puncture apparatus according to the disclosure is not limited to the embodiments.

According to the disclosure here, for example, when the implant is indwelled, the patient burden is minimal, the patient safety is high, and the operator safety is also high.

That is, when an operator punctures the living body using the puncture needle, the operator can conduct a manual procedure while visually recognizing the ultrasonic marker by observing an ultrasonic image, and the distal end of the puncture needle is guided to the ultrasonic marker or the puncture needle protrudes from a position of the ultrasonic marker or a position in the vicinity of the ultrasonic marker so the distal end of the puncture needle can be guided to the ultrasonic probe. Accordingly, the puncture needle can be prevented from puncturing the urethra, and it is possible for an operator himself or herself to prevent the puncture needle from puncturing his or her finger tip.

It is thus possible to indwell the implant using low invasive manual procedures.

The detailed description above describes a puncture apparatus disclosed by way of example. The disclosure is not limited, however, to the precise embodiment and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A puncture apparatus comprising:
a puncture needle configured to puncture biological tissues;
a longitudinal-shaped vaginal insertion member configured for insertion into a vagina of a living being;
a guiding member;
interlock means for interlocking the vaginal insertion member with the guiding member;
an ultrasonic probe that is provided on one side of either a distal portion of the vaginal insertion member or the guiding member to transmit and receive ultrasonic waves;
an ultrasonic marker that is provided on the other side of either the distal portion of the vaginal insertion member or the guiding member to be able to recognize an ultrasonic image which is obtained from the ultrasonic waves transmitted and received from the ultrasonic probe; and
supporting means for movably supporting the puncture needle to guide the distal end of the puncture needle from a side of the ultrasonic probe toward the ultrasonic marker.

2. The puncture apparatus according to claim 1,
wherein the guiding member is provided to be movable in a longitudinal direction of the vaginal insertion member relative to the vaginal insertion member and to be rotatable.

3. The puncture apparatus according to claim 2,
wherein the supporting means includes heading direction changing means for changing a direction in which the puncture needle advances in such a manner that the puncture needle faces a direction of the ultrasonic marker when the guiding member moves in the longitudinal direction of the vaginal insertion member relative to the vaginal insertion member or moves rotationally.

4. The puncture apparatus according to claim 3,
wherein the ultrasonic probe is provided in the distal portion of the vaginal insertion member, and the ultrasonic marker is provided in the guiding member.

5. The puncture apparatus according to claim 1,
wherein the ultrasonic probe is provided in the guiding member, and the ultrasonic marker is provided in the distal portion of the vagina insertion member.

6. The puncture apparatus according to claim 5,
wherein the ultrasonic probe is rotatably supported by the guiding member.

7. A puncture apparatus comprising:
a puncture needle configured to puncture biological tissues;
a longitudinal-shaped vaginal insertion member configured to be inserted into a vagina of a living being;
a guiding member;
interlock means for interlocking the vaginal insertion member with the guiding member;
an ultrasonic probe that is provided on one side of either a distal portion of the vaginal insertion member or the guiding member to transmit and receive ultrasonic waves;
an ultrasonic marker that is provided on the other side of either the distal portion of the vaginal insertion member or the guiding member to be able to recognize an ultrasonic image which is obtained from the ultrasonic waves transmitted and received from the ultrasonic probe; and
supporting means for movably supporting the puncture needle to guide the distal end of the puncture needle from a side of the ultrasonic marker toward the ultrasonic probe.

8. The puncture apparatus according to claim 7,
wherein the guiding member is provided to be movable in the longitudinal direction of the vaginal insertion member relative to the vaginal insertion member.

9. The puncture apparatus according to claim 7,
wherein the guiding member is provided to be movable in the longitudinal direction of the vaginal insertion member relative to the vaginal insertion member and to be rotatable.

10. The puncture apparatus according to claim 7,
wherein the supporting means has a through-hole or a groove into which the puncture needle is inserted.

11. The puncture apparatus according to claim 7,
wherein the ultrasonic marker is provided in a distal portion of the puncture needle to be able to recognize an ultrasonic image which is obtained from ultrasonic waves transmitted and received from the ultrasonic probe.

12. The puncture apparatus according to claim 1,
wherein the supporting means has a through-hole or a groove into which the puncture needle is inserted.

13. The puncture apparatus according to claim 1,
wherein the ultrasonic marker is provided in a distal portion of the puncture needle to be able to recognize an ultrasonic image which is obtained from ultrasonic waves transmitted and received from the ultrasonic probe.

14. The puncture apparatus according to claim 1,
wherein the ultrasonic marker includes a plurality of small concaves and convexes formed on the distal portion of the puncture needle.

15. The puncture apparatus according to claim 7,
wherein the ultrasonic marker includes a plurality of small concaves and convexes formed on the guiding member.
